(19) United States
(12) Patent Application Publication
(10) Pub. No.: US 2007/0010507 A1
(43) Pub. Date: Jan. 11, 2007

(54) Compounds useful in the therapy of alzheimer's disease and formulations containing them

(76) Inventor: Ezio Bombardelli, Groppello Cairoli (IT)

Correspondence Address: YOUNG & THOMPSON, 745 SOUTH 23RD STREET, 2ND FLOOR, ARLINGTON, VA, 22202, US

(21) Appl. No.: 10/558,403

(22) PCT Filed: May 26, 2005
(86) PCT No.: PCT/EP04/05644

§ 371 Date: Apr. 28, 2006

(30) Foreign Application Priority Data
May 30, 2003 (IT) MI2003A001098

(51) Int. Cl. A61K 31/55; A61K 31/473; A61K 31/4745; A61K 31/407; A61K 31/13; A61K 31/12

(52) U.S. Cl. 514/214.01; 514/297; 514/411; 514/283; 514/690; 514/305; 514/662

(57) ABSTRACT

Floroglucino salts, in particular salts of tetrahydro- and octahydro-hyperforin or adhyperforin derivates and of colupon with acetylcholinesterase-inhibiting alkaloids and methods for the preparation thereof. The salts are useful for the treatment of depression and Alzheimer's disease can be administered as conventional pharmaceutical formulations or as controlled-release transdermal preparations.

### FIELD OF THE INVENTION

The present invention concerns floroglucinol salts with acetylcholinesterase-inhibiting alkaloids and methods for the preparation thereof.

### TECHNOLOGICAL BACKGROUND

Amyloid peptide Aβ1-42, a product of transformation of Alzheimer Precursor Protein (APP), plays as key role in the appearance of Alzheimer's disease (Nature Medicine, 3, 28-29, 1997; Science, 275, 630-1, 1997).

APP antisense mutations in patients with inherited Alzheimer induce an increase in the release of Aβ1-42. Presenilin 1 and 2 mutations also increase Aβ1-42 (Nature Medicine 1996, 2, 854-870).

Transgenic mice showing a mutated APP excess develop age-dependent β-amyloid deposits and have cognitive disorders (Science, 1996, 274, 99-102; Nature, 373, 523-27, 1995).

The proteolytic cleavage of pathogenic β-amyloid is mediated by β- and γ-secretase. α-Secretase transforms APP into a soluble, non-pathogenic form, reducing amyloid deposit vascular at the cerebral level. α-Secretase is stimulated by acetylcholine, with the mediation of m1 and m3 muscarinic receptors (Science 1992, 258, 304-307). The cell mediator is protein kinase C (PKC) (Proc. Natl. Acad. Sci. USA 1990, 87, 6003-6006).

Some acetylcholinesterase-inhibiting alkaloids proved to be useful in the therapy of Alzheimer's disease, apparently through indirect stimulation of a-secretase. By way of example, galantamine inhibits acetylcholinesterase causing an increase in acetylcholine. This activates PKC through M1 and M3 muscarinic receptors, thereby inducing an increase in α-secretase and a consequent decrease in pathogenic amyloid.

Physostigmine, huperzine and tacrine are other examples of acetylcholinesterase-inhibiting alkaloids. NMDA antagonistic alkaloids (e.g. memantine), nootropics, cholinergics, glutamine antagonists, serotoninergics, MAO inhibitors, PKC activators, muscarinic agonists may also be conveniently used to this purpose.

In view of what stated above, the use of substances which selectively activate PKC, and particularly PKC-γ, which is present in nervous cells only, is an important approach in the therapy of Alzheimer's disease. Furthermore, all substances either directly or indirectly stimulating a-secretase are important inhibitors of pathogenic β-amyloid and therefore suitable for the therapy of Alzheimer's disease.

Hyperforin salts with anticholinesterase alkaloids were recently disclosed in WO/99/41220 as particularly interesting for the treatment of Alzheimer's disease. It has however been subsequently observed that the pharmacological activity of said salts decreases in vivo due to metabolic reactions.

### DISCLOSURE OF THE INVENTION

It has now been found that floroglucinol salts, in particular salts of tetrahydro- and octahydro-hyperforin or adhyperforin derivatives and of colupolon with alkaloids, are metabolically stable and induce higher α-secretase activity compared to the single compounds and the salts described in WO/99/41220.

The compounds according to the present invention have general formula (I)
[A] [B] (I)
in which [A] is the anion of formula (II) 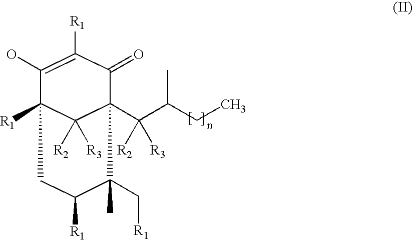
wherein:
n is 0 or 1;
when R₁ is iso-pentenyl, R₂ is hydroxyl and R₃ is hydrogen;
when R₁ is iso-pentyl, R₂ is hydroxyl and R₃ is hydrogen, or R₂ and R₃ are taken together to form a carbonyl;
   or [A] is the anion of colupolon of formula (III)

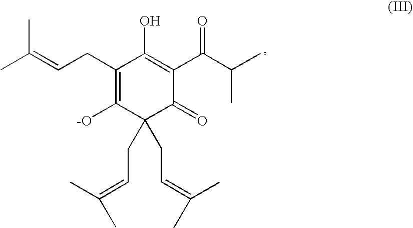

formulae (II) and (III) also including the corresponding tautomeric forms;

and [B] is the cation of an alkaloid selected from: anticholinesterase agents, NMDA-antagonists, nootropics, cholinergics, glutamine antagonists, serotoninergics, MAO inhibitors, PKC activators, muscarinic agonists, vincamine, apovincamine, chinidine and eseroline.

Preferred anticholinesterase agents are galantamine, physostigmine, huperzine and tacrine; a preferred NMDA-antagonist is memantine.

Furthermore, particularly preferred are the following salts:
octahydrohyperforin salt with galantamine;
octahydrohyperforin salt with physostigmine;
octahydrohyperforin salt with memantine;
octahydrohyperforin salt with vincamine;
octahydrohyperforin salt with apovincamine;
octahydrohyperforin salt with chinidine;
octahydrohyperforin salt with eseroline;
octahydrohyperforin salt with tacrine;
galantamine salt with colupolon.

Hyperforin and adhyperforin saturated derivatives can be obtained by direct reduction of hexane or ether extracts enriched in the Hypericum perforatum flowering tops.

In particular, the tetrahydro-derivatives can be obtained by reduction with hydrides, whereas the octahydro-derivatives can be obtained by chemoselective reduction of the isoprene chains by catalytic hydrogenation, as described WO 03/09114.

Hyperforin derivatives and colupolon are salified with the above mentioned alkaloids according to conventional methods, well known to those skilled in the art.

The salts according to the present invention are stable crystalline products having strong antidepressive and anti-Alzheimer actions. The effect of the compounds of the invention on α-secretase-induced APPs release was determined in the culture medium of a neuroblastoma cell line (SH-SY5Y) according to what reported by Galbete J. L. et al. in Biochem. 348,307-313, 2000. The results reported in the following Table show that the tested compounds activate α-secretase-mediated APP metabolism inducing an increase in APPs secreted in the culture medium.

TABLE

| Substances | APPs % |
|---|---|
| Control | 100 |
| Hyperforin (10 μM) | 296 |
| Octahydrohyperforin (10 μM) | 1383 |
| Galantamine | 302 |
| Galantamonium hyperforinate | 773 |
| Galantamonium octahydrohyperforinate (10 μM) | 3652 |
| Mentamine octahydrohyperforinate (10 μM) | 3221 |

It has also been observed that the compounds of the invention are active both as acetylcholinesterase inhibitors and protein kinase C activators.

The present invention, therefore, also relates to pharmaceutical compositions containing the salts of formula (I). Said compositions will be in the form of soft-gelatin capsules, hard-gelatin capsules, tablets, suppositories and controlled-release formulations, prepared according to conventional methods, such as those reported in Remington's Pharmaceutical Sciences Handbook, XVII Ed., Mack Pub., N.Y., U.S.A.

Preferred pharmaceutical forms are hard- and soft-gelatin capsules, tablets and transdermal plasters. In the latter case, the controlled release of the compounds of the invention can be obtained by placing the plaster in a proximal area to cerebral carotids arterial derivations. The dosages of compound in the formulations will range from 10 to 100 mg/dose/daily.

The invention will be illustrated in further detail by the following examples.

### EXAMPLE I

### Octahydrohyperforin Salt with Galantamine

3.4033 g of octahydrohyperforin are dissolved in 10 ml of MeOH at room temperature. The solution is added with 1.72 g of galantamine dissolved in 5 ml of MeOH. The solution is added with water until slight turbidity and left to stand in refrigerator overnight. Galantamine octahydrohyperforinate is obtained, having the following physico-chemical characteristics:

¹H-NMR (300 MHz CDCl₃): δ 6.72 (1H, d, J=8.5 Hz, CH), 6.68 (1H, d, J=8.5 Hz, CH), 6.07 (1H, s, CH), 4.66 (1H, m, CH), 4.18 (1H, m, CH), 3.88 (3H, s, CH₃), 3.79 (2H, d, J=15.0 Hz, CH₂), 3.38 (1H, t, J=13.6 Hz, CH), 3.14 (1H, td, J=13.6, 4.0 Hz, CH), 2.73 (1H, dd, J=16.0 and 4.8 Hz, CH), 2.48 (3H, s, CH₃), 1.93-1.00 (22H, m, H-4, H-11, CH₂-5, CH₂-15, CH₂-16, CH₂-17, CH₂-21, CH₂-22, CH₂-26, CH₂-27, CH₂-31, CH₂-32), 1.00-0.80 (24H, d, CH₃-19, CH₃-20, CH₃-24, CH₃-25, CH₃-29, CH₃-30, CH₃-34, CH₃-35), 1.20, 1.06 (6H, d, J=6.3 Hz, CH₃-12, CH₃-13), 0.91 (3H, s, CH₃-14).

¹³C-NMR (75 MHz CDCl₃): δ 211.41, 208.09, 146.20, 144.85, 133.12, 128.22, 127.31, 126.68, 122.77, 121.36, 111.80, 88.87, 62.29, 60.35, 56.18, 53.81, 49.29, 48.30, 42.16, 42.06, 41.39, 41.25, 40.34, 37.82, 37.75, 37.60, 33.68, 33.30, 30.11, 29.88, 29.04, 28.49, 28.38, 28.16, 26.90, 23.98, 22.96, 22.88, 22.82, 22.72, 22.68, 22.61, 21.88, 21.16, 20.89, 14.82.

ESIMS m/z 288 [Galantamine+H⁺] (100), 543 [Octahydrohyperforin-H⁺] (100), 1109 [2(Octahydrohyperforin-H⁺)+Na⁺] (92).

### EXAMPLE II

### Octahydrohyperforin Salt with Physostigmine

Following the procedure described in Example I, physostigmine base is salified with an octahydrohyperforin equivalent. Physostigmine octahydrohyperforinate is obtained, having the following physico-chemical and spectroscopic characteristics:

¹H-NMR (300 MHz CDCl₃): δ 6.86 (1H, dd, J=8.3 and 2.2 Hz, CH), 6.81 (1H, d, J=2.2 Hz, CH), 6.41 (1H, d, J=8.3 Hz, CH), 4.28 (2H, s, CH₂), 2.98 (3H, s, CH₃), 2.91 (2H, d, J=4.8 Hz, CH₂), 2.86 (3H, m, CH₃), 2.60 (3H, m, CH₃), 1.48 (3H, m, CH₃), 1.93-1.00 (22H, m, H-4, H-11, CH₂-5, CH₂-15, CH₂-16, CH₂-17, CH₂-21, CH₂-22, CH₂-26, CH₂-27, CH₂-31, CH₂-32), 1.00-0.80 (24H, d, CH₃-19, CH₃-20, CH₃-24, CH₃-25, CH₃-29, CH₃-30, CH₃-34, CH₃-35), 1.20, 1.06 (6H, d, J=6.3 Hz, CH₃-12, CH₃-13), 0.91 (3H, s, CH₃-14).

³C-NMR (75 MHz CDCl₃): δ 211.73, 208.47, 156.37, 149.17, 144.02, 137.03, 121.19, 121.13, 116.47, 107.38, 97.82, 77.71, 77.28, 76.86, 53.44, 53.21, 49.07, 42.20, 41.12, 40.33, 37.89, 37.72, 37.56, 33.71, 29.95, 29.10, 28.51, 28.35, 28.19, 27.95, 27.27, 26.90, 24.05, 22.99, 22.96, 22.90, 22.85, 22.79, 22.70, 22.63, 21.93, 21.31, 20.88, 14.74.

ESIMS m/z 276 [Physostigmine+H⁺] (100), [2Physostigmine+Na⁺] (34), 543 [Octahydrohyperforin-H⁺] (100), 1109 [2(Octahydrohyperforin-H⁺)+Na⁺] (23).

### EXAMPLE III

### Octahydrohyperforin Salt with Memantine

Following the procedure described in Example I, memantine base is salified with an octahydrohyperforin equivalent. Memantine octahydrohyperforinate is obtained, having the following physico-chemical and spectroscopic characteristics:

¹H-NMR (300 MHz CDCl₃): δ 2.38, 2.21, 1.19, 1.12, 1.0. 0.97, 0.96, 0.95, 0.93, 0.92, 0.90, 0.88.

¹³C-NMR (75 MHz CDCl₃): δ 211.72, 121.11, 60.03, 50.52, 50.16, 48.79, 43.06, 42.51, 42.19, 41.01, 40.36, 37.86, 37.60, 33.67, 32.90, 30.45, 30.36, 30.13, 29.96, 29.05, 28.61, 28.19, 26.98, 24.00, 22.98, 22.87, 22.75, 22.66, 22.61, 21.98, 21.26, 20.87, 14.83.

ESIMS m/z 180 [Memantine+H⁺] (100), [M+H⁺] (34), 543 [Octahydrohyperforin-H⁺] (100), 1109.

### EXAMPLE IV

### Octahydrohyperforin Salt with Vincamine

Following the procedure described in Example I, vincamine is salified with an octahydrohyperforin equivalent, increasing the MeOH volumes compared with the other alkaloids. Vincamine octahydrohyperforinate is obtained, having the following physico-chemical and spectroscopic characteristics:

¹H-NMR (300 MHz CDCl₃): δ 7.53 (1H, m, CH)-7.18 (3H, m, CH)-14 1.93-1.00 (22H, m, H-4, H-11, CH₂-5, CH₂-15, CH₂-16, CH₂-17, CH₂-21, CH₂-22, CH₂-26, CH₂-27, CH₂-31, CH₂-32), 1.00-0.80 (24H, d, CH₃-19, CH₃-20, CH₃-24, CH₃-25, CH₃-29, CH₃-30, CH₃-34, CH₃-35), 1.20, 1.06 (6H, d, J=6,3 Hz, CH₃-12, CH₃-13), 0.91 (3H, s, CH₃-14).

¹³C-NMR (75 MHz CDCl₃): δ 207.42, 174.47, 134.63, 128.92, 122.37, 121.62, 120.79, 118.85, 110.72, 105.99, 82.09, 59.62, 54.60, 51.31, 49.57, 44.86, 44.50, 42.40, 41.08, 40.22, 37.83, 37.68, 37.47, 35.57, 33.69, 29.83, 29.15, 29.07, 28.50, 28.40, 28.16, 26.91, 24.96, 23.78, 22.96, 22.93, 22.87, 22.79, 22.69, 22.63, 21.81, 21.13, 20.80, 20.52, 16.95.

ESIMS m/z 355 [Vincamine+H⁺] (100), 543 [Octahydrohyperforin-H⁺] (100).

### EXAMPLE V

### Octahydroadhyperforin Salt with Apovincamine

Following the procedure described in Example I, apovincamine base is salified with an octahydrohyperforin equivalent. Apovincamine octahydrohyperforinate is obtained, having the following physico-chemical and spectroscopic characteristics:

¹H-NMR (300 MHz CDCl₃): δ 7.51 (1H, m, CH), 7.22 (1H, m, CH), 6.18 (1H, m, CH), 5.07 (4H, m, CH), 3.99 (3H, m, CH), 1.93-1.00 (22H, m, H-4, H-11, CH₂-5, CH₂-15, CH₂-16, CH₂-17, CH₂-21, CH₂-22, CH₂-26, CH₂-27, CH₂-31, CH₂-32), 1.00-0.80 (24H, d, CH₃-19, CH₃-20, CH₃-24, CH₃-25, CH₃-29, CH₃-30, CH₃-34, CH₃-35), 1.20, 1.06 (6H, d, J=6,3 Hz, CH₃-12, CH₃-13), 0.91 (3H, s, CH₃-14).

¹³C-NMR (75 MHz CDCl₃): δ 211.4, 191.3, 184.6, 163.7, 134.7, 128.8, 128.6, 127.7, 122.8, 120.9, 118.7, 112.9, 108.6, 82.7, 61.5, 56.3, 51.3, 47.7, 41.5, 40.5, 38.2, 37.9, 37.7, 33.9, 30.5, 29.6, 28.7, 28.3, 28.1, 27.1, 23.3, 23.1, 23.0, 22.8, 22.7, 22.4, 22.0, 14.0.

ESIMS m/z 337 [Apovincamine+H⁺] (100), 543 [Octahydrohyperforin-H⁺] (100).

### EXAMPLE VI

### Octahydroadhyperforin Salt with Chinidine

Following the procedure described in Example I, chinidine base is salified with an octahydrohyperforin equivalent. Chinidine octahydrohyperforinate is obtained, having the following physico-chemical and spectroscopic characteristics:

¹H-NMR (300 MHz CDCl₃): δ 8.78 (1H, d(4.6), CH), 7.95 (1H, d(9.2), CH), 7.64 (1H, d(4.6), CH), 7.34 (1H, dd(9.2 and 2.6), CH), 7.30 (1H, m, CH), 6.05 (1H, m, CH), 5.14 (6H, m), 3.86 (4H, m), 3.18 (5H, m), 1.93-1.00 (22H, m, H-4, H-11, CH₂-5, CH₂-15, CH₂-16, CH₂-17, CH₂-21, CH₂-22, CH₂-26, CH₂-27, CH₂-31, CH₂-32), 1.00-0.80 (24H, d, CH₃-19, CH₃-20, CH₃-24, CH₃-25, CH₃-29, CH₃-30, CH₃-34, CH₃-35), 1.20, 1.06 (6H, d, J=6,3 Hz, CH₃-12, CH₃-13), 0.91 (3H, s, CH₃-14).

³C-NMR (75 MHz CDCl₃): δ 211.4, 191.3, 184.6, 158.2, 147.7, 147.1, 144.5, 126.8, 121.3, 101.7, 82.7, 61.5, 56.2, 51.3, 47.7, 41.5, 40.5, 38.2, 37.9, 37.7, 33.9, 30.5, 29.6, 28.7, 28.3, 28.1, 27.1, 23.3, 23.1, 23.0, 22.8, 22.7, 22.4, 22.0, 14.0.

ESIMS m/z 325 [Chinidine+H⁺] (100), 543 [Octahydrohyperforin-H⁺] (100).

### EXAMPLE VII

### Octahydrohyperforin Salt with Eseroline

Following the procedure described in Example I, eseroline base is salified with an octahydrohyperforin equivalent. Eseroline octahydrohyperforinate is obtained, having the following physico-chemical and spectroscopic characteristics:

¹H-NMR (300 MHz CD₃OD): δ 6.68 (1H, dd, J=9.8 and 2.7 Hz, CH), 6.66 (1H, s, CH), 6.55 (1H, d, J=7.8 Hz, CH), 3.38 (1H, m, CH), 3.25 (2H, m, CH₂), 3.07 (3H, s, CH₃), 2.85 (3H, s, CH₃), 1.93-1.00 (22H, m, H-4, H-11, CH₂-5, CH₂-15, CH₂-16, CH₂-17, CH₂-21, CH₂-22, CH2-26, CH₂-27, CH₂-31, CH₂-32), 1.00-0.80 (24H, d, CH₃-19, CH₃-20, CH₃-24, CH₃-25, CH₃-29, CH₃-30, CH₃-34, CH₃-35), 1.20, 1.06 (6H, d, J=6.3 Hz, CH₃-12, CH₃-13), 0.91 (3H, s, CH₃-14).

¹³C-NMR (75 MHz CDCl₃): δ 207.36, 121.60, 53.26, 49.59, 42.41, 41.59, 41.09, 40.20, 37.81, 37.67, 37.46, 33.69, 29.82, 29.05, 28.49, 28.40, 28.17, 26.90, 23.76, 22.96, 22.93, 22.87, 22.79, 22.68, 22.63, 21.82, 21.11, 20.80, 15.22.

ESIMS m/z 219 [Eseroline+H⁺] (100), 1111 [2Octahydrohyperforin+Na⁺] (72), 567 [Octahydrohyperforin+Na⁺] (27), 543 [Octahydrohyperforin-H⁺] (100), 1109 [2(Octahydrohyperforin-H⁺)+Na⁺] (29).

### EXAMPLE VIII

### Octahydrohyperforin Salt with Tacrine

Following the procedure described in Example I, tacrine base is salified with an octahydrohyperforin equivalent. Tacrine octahydrohyperforinate is obtained, having the following physico-chemical and spectroscopic characteristics:

¹H-NMR (300 MHz CD₃OD): δ 8.37 (1H, d, J=8.8 Hz, CH), 7.91 (1H, t, J=7.8 Hz, CH), 7.78 (1H, d, J=8.1 Hz, CH), 7.65 (1H, t, J=8.0 Hz, CH), 3.05 (2H, t, J=5.8 Hz, CH₂), 2.68 (2H, t, J=5.8 Hz, CH₂), 2.46 (2H, m, CH₂), 2.02 (2H, m, CH₂), 93-1.00 (22H, m, H-4, H-11, CH₂-5, CH₂-15, CH₂-16, CH₂-17, CH₂-21, CH₂-22, CH₂-26, CH₂-27, CH₂-31, CH₂-32), 1.00-0.80 (24H, d, CH₃-19, CH₃-20, CH₃-24, CH₃-25, CH₃-29, CH₃-30, CH₃-34, CH₃-35), 1.20, 1.06 (6H, d, J=6.3 Hz, CH₃-12, CH₃-13), 0.91 (3H, s, CH₃-14).

¹³C-NMR (75 MHz CDCl₃): δ 212.51, 210.48, 155.50, 152.00, 138.00, 132.67, 125.97, 123.70, 120.21, 115.58, 109.09, 60.47, 48.40, 42.17, 41.61, 41.34, 40.57, 40.38, 40.01, 38.20, 37.84, 37.62, 33.76, 30.17, 29.12, 28.72, 28.41, 28.12, 26.92, 24.04, 22.98, 22.90, 22.88, 22.80, 22.73, 22.61, 22.46, 22.17, 21.86, 21.21, 21.10, 14.73.

ESIMS m/z 199 [Tacrine+H⁺] (100), 543 [Octahydrohyperforin-H⁺] (100).

### EXAMPLE IX

### Galantamine Salt with Colupolon

Following the procedure described in Example I, galantamine base is salified with an equivalent of colupolon. The salt has the following physico-chemical and spectroscopic characteristics:

¹H-NMR (300 MHz CDCl₃): δ 6.73 (1H, d, J=7.6 Hz, CH), 6.69 (1H, d, J=8.5, CH), 6.07 (1H, s, CH), 5.18 (1H, s, broadened singlet, CH), 4.84 (1H, broadened singlet, CH), 4.67 (1H, t, J=3.0, CH), 4.28-3.87 (2H, d, J=15.0, CH₂), 4.19 (1H, m, CH), 3.88 (3H, s, CH₃), 3.46-3.22 (2H, td-m, J=14.0, 2.0, CH₂), 2.74-2.30 (2H, dd-dd, J=16.1, 4.4-5.1, 2.4, CH₂), 2.53 (3H, s, CH₃), 2.41 (2H, d, J=7.4, CH₂), 2.15-1.76 (2H, td-m, J=14.0, 3.1, CH₂), 1.81 (3H, s, CH₃), 1.73 (3H s, CH₃), 1.62 (3H, s, CH₃), 1.59 (3H, s, CH₃), 1.17 (3H, d, J=6.7, CH₃), 2.68-2.55 (2H, m, CH₂).

¹³C-NMR (75 MHz CDCl₃): δ 212.33, 203.98, 200.59, 182.63, 146.32, 145.2, 134.45, 133.14, 128.73, 126.20, 123.19, 122.94, 119.23, 111.96, 88.84, 59.88, 56.21, 53.63, 48.10, 40.95, 37.78, 30.13, 26.06, 26.02, 19.09, 18.18, 18.10.

ESIMS m/z 288 [Galantamine+H⁺] (100), 597 [2Galantamine+Na⁺] (22), 399 [Colupulone-H⁺] (100).

### EXAMPLE X

### Formulation Containing Galantamine Octahydrohyperforinate

10 mg of galantamine octahydrohyperforinate are diluted with 80 mg of lactose, 5 mg of magnesium stearate and distributed into a hard gelatin capsule.

### EXAMPLE XI

### Transdermal Plaster Containing Galantamine Octahydrohyperforinate

An adhesive suspension based on acrylate (Durotak 387-22-87) was prepared containing 10% of galantamine octahydrohyperforinate and 15% of N-methyl-2-pyrrolidone, to obtain the give medicament in the form of gel. This gel was deposited onto a polyester film (S2016) by means of a coating apparatus. After drying at 80° C. for 10 min., a polyester film (Scotchpack 1109) was laminated on the dried medicament. The resulting sheet was cut into plasters of the desired size.

### EXAMPLE XII

### Transdermal Plaster Containing Mentamine Octahydrohyperforinate

0.050 g of mentamine octahydrohyperforinate were suspended in 10 g of a medical silicone adhesive (Dow Corning MD7-4502) to give a gel of the medicament. The gel was deposited with solvent onto a polyester film (S2016) by means of a coating apparatus. After drying at 80° C. for 10 minutes, a polyester film (Scotchpack 1109) was laminated onto the gel of dried medicament and the resulting sheet was cut into plasters of the desired size.

1. Salts of general formula (I)
[A] [B] (I)
in which [A] is the anion of formula (II) 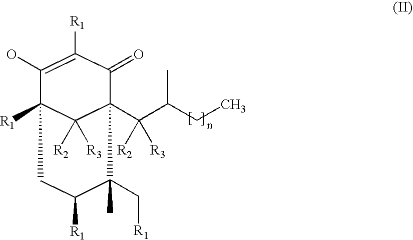
wherein:
n is 0 or 1;
when R, is iso-pentenyl, R2 is hydroxyl and R3 is hydrogen;
when R, is iso-pentyl, R2 is hydroxyl and R3 is hydrogen, or R2 and R3 are taken together to form a carbonyl;
or[A] is the anion of colupolon of formula (III) 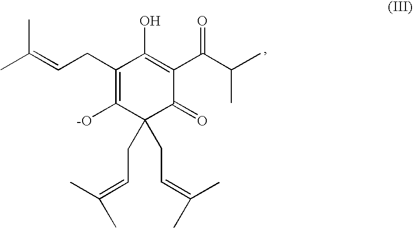
formulae (II) and (III) also including the corresponding tautomeric forms; and [B] is the cation of an alkaloid selected from: anticholinesterase agents, NMDA-antagonists, nootropics, cholinergics, glutamine antagonists, serotoninergics, MAO inhibitors, PKC activators, muscarinic agonists, vincamine, apovincamine, chinidine and eseroline.

2. Salts as claimed in claim 1, wherein the anticholinesterase alkaloid is selected from galantamine, physostigmine, huperzine and tacrine.

3. Salts as claimed in claim 1, wherein the NMDA-antagonist alkaloid is memantine.

4. A compound selected from:
octahydrohyperforin salt with galantamine;
octahydrohyperforin salt with physostigmine;
octahydrohyperforin salt with memantine;
octahydrohyperforin salt with vincamine;
octahydrohyperforin salt with apovincamine;
octahydrohyperforin salt with chinidine;
octahydrohyperforin salt with eseroline;
octahydrohyperforin salt with tacrine;
galantamine salt with colupolon.

5. Salts as claimed in claim 1 as medicaments.

6. The use of the salts as claimed in claim 1 for the preparation of medicaments for the therapy of Alzheimer's disease.

7. The use of the salts as claimed in claim 1 for the preparation of medicaments for the therapy of depression.

8. Pharmaceutical compositions containing a salt as claimed in claim 1 in admixture with suitable excipients and/or carriers.

9. Capsules containing a salt as claimed in claim 1.

10. Transdermal plasters containing the salts as claimed in claim 1.
